(19) United States
(12) Patent Application Publication
(10) Pub. No.: US 2016/0228698 A1
(43) Pub. Date: Aug. 11, 2016

(54) TREATMENT DEVICE FOR THE SKIN USING RADIO-FREQUENCY ELECTRIC CURRENT

(71) Applicant: KONINKLIJKE PHILIPS N.V., Eindhoven (NL)

(72) Inventors: MARGARET RUTH HORTON, EINDHOVEN (NL); JONATHAN ALAMBRA PALERO, EINDHOVEN (NL); MARTIN JURNA, EINDHOVEN (NL); BABU VARGHESE, EINDHOVEN (NL)

(21) Appl. No.: 15/022,330

(22) PCT Filed: Sep. 17, 2014
(86) PCT No.: PCT/EP2014/069781

§ 371 Date: Mar. 16, 2016

(30) Foreign Application Priority Data
Sep. 19, 2013 (EP) 13185070.3

(51) Int. Cl. A61N 1/30; A61N 1/40; A61N 1/04

(52) U.S. Cl. CPC A61N 1/30; A61N 1/0432; A61N 1/0448; A61N 1/403

(57) ABSTRACT

The invention provides a non-invasive r.f. current treatment device to be used with an agent or treatment substance (20), such as an ionic salt solution. The substance is transported using an electric field generated by the device, such as in iontophoresis, to move the treatment substance (20) into the skin so that the skin's electrical transmission of the r.f-current is increased. The path of any r.f. current is influenced by the distribution of the treatment substance (20) in the skin. Applying r.f. current causes heating in skin tissues coinciding with the path of the r.f. current, allowing skin tissue to be treated that is either coincident with the r.f. current path or immediately adjacent thereto. The region of skin treated with r.f. current may be the upper tissue layers. This renders superfluous the conventional hydration step necessary for r.f. skin preparation, which is poorly controllable and highly variable depending on the anatomic site and the individual being treated. The lowered skin impedance enables well-defined r.f. delivery to skin tissues, and a high degree of control over the r.f. energy delivery depth profile. The invention also makes it possible to treat deeper layers, using the r.f. current, than traditional techniques due to the high penetration of the treatment substance (20) caused by application of the electric field.

### FIELD OF THE INVENTION

The invention relates generally to a treatment device for the skin, in particular to a non-invasive treatment device for skin treatment by using a radio-frequency (r.f.) electric current.

### BACKGROUND OF THE INVENTION

Various forms of electromagnetic radiation, particularly laser light beams, have been used on the skin for many years for a variety of treatments, such as hair removal, skin rejuvenation to reduce wrinkles, and the treatment of conditions like acne, actinic keratosis, blemishes, scar tissue, discoloration, vascular lesions, acne treatment, cellulite and tattoo removal. Most of these treatments rely on photothermolysis, where a treatment location is targeted by the treatment radiation. For example, to treat wrinkles, the dermis layer is damaged by heating (thermolysis) to induce a wound response while minimizing damage to the epidermis.

Radio-frequency (usually abbreviated as r.f. or rf) energy has also been used for skin rejuvenation and skin tightening in both the professional and home-use aesthetic device market. Compared to laser treatments, r.f. devices have a substantially, relatively lower cost price and can provide larger-volume deep-tissue contraction. Additionally, r.f. energy dissipation does not rely on absorption of light by chromophores, so that tissue pigmentation does not interfere with the delivery of energy. The advantages of r.f. over laser treatments, and some embodiments of known r.f. devices, are described in the article “Radio Frequency Energy for Non-invasive and Minimally Invasive Skin Tightening”, by R. Stephen Mulholland, Clin Plastic Surg 38 (2011) 437-448.

For skin treatments, the energy applied to the skin is usually maximized so that treatment times are kept reasonably short and the skin rejuvenation results are optimized. Unfortunately, r.f. treatment efficacy is highly dependent on the electrochemical properties of the skin, which are highly variable at different sites of the body, and among different people. Such differences in skin properties are described in the article “The electric resistivity of human tissues (100 Hz-10 MHz): a meta-analysis of review studies” by Faes, van der Meij, de Munck and Heethaar, Physiological Measurements, 20 (1999) R1-R10.

The basic principle of r.f. energy delivery via the skin surface to tissue is that an alternating current is applied in a closed circuit with the skin. The r.f. energy is dissipated as thermal energy primarily due to intramolecular vibrations. Tissue impedance directly affects the extent of the heating: r.f. propagates more easily through tissues with high conductivity (low electrical resistance), while tissues with high electrical resistance (high impedance, low conductance) are poor conductors of r.f. energy.

The thermal effects generated from r.f. energy applied through electrodes at the surface of the skin range from sub-epidermal tissue contraction to surface ablation, depending on electrode configuration (size, inter-electrode distance, proximity to skin) and energy delivered (frequency, power, duration). These effects have different desired effects on the skin: surface ablation is used for skin rejuvenation of the epidermis, or to enable the penetration of substances, while sub-epidermal tissue contraction is primarily used for skin tightening purposes and to stimulate new collagen synthesis.

The local electrical conductivity of tissue strongly influences the heat dispersion in the tissue and therefore the outcome of the r.f. treatment. Therefore, one of the known disadvantages of r.f. treatments is the inconsistency of tissue heating among different individuals at different skin sites. In particular the uppermost layers of the skin, primarily the stratum corneum, is the entry point for the r.f. energy delivered into the skin from the surface. Insufficient conductance within this layer can lead to surface burns or undesired accumulation of energy in the upper skin layers and prevention of r.f. energy conductance to deeper target tissue layers. Thus, variable properties of the skin negatively influence the targeting, reliability and safety of the r.f. treatment.

Burning or charring of the skin in the immediate vicinity of the electrodes at these higher energy levels is a major problem. This is particularly difficult to control with invasive devices requiring an electrode to be implanted in the body, such as the Thermage device described in the Mulholland article. The variation in skin properties means that calibration must be done between different people and between different treatment sites. The traditional approach described in the article by Mulholland is to prevent burning or charring by monitoring the impedance and the temperature, and to cut-off the r.f. energy when the temperature at the electrodes is too high. However, this will result in a longer treatment time for the person being treated, and a large variability in efficacy at different treatment locations on the body.

It is known from the field of tumor ablation using r.f. energy that charring around the electrodes may occur when the energy is maximized to optimize ablation. This is described in the article “Radio frequency Ablation of Liver Tumors: A Novel Needle Perfusion Technique Enhances Efficiency” by P. Abitabile and C. A. Maurer, J of Surgical Research, 159 (2010) 532-537. The article provides two improvements to optimize the ablation—inject saline directly into the tumor or continuously rinse the skin area around the electrodes with saline. However, injecting saline is an invasive procedure requiring medical expertise.

EP 1 568 395 A1 discloses a treatment method and apparatus for providing a substance to be absorbed onto a surface of a patient's skin. The method includes applying the substance onto the surface of the patient's skin by way of a probe head that provides, at the same time, bursts of electrical pulses and vibrations to the skin surface. The vibrations are applied to the skin surface at substantially a same frequency rate, a first harmonic of the same frequency rate, and/or a second harmonic of the same frequency rate as a burst rate of the electrical pulses applied to the skin surface. In an embodiment a D.C. current is added to the electrical pulses to induce an additional iontophoresis effect. In a further embodiment additional heating of the skin is effected by means of radio frequency electrodes in order to increase the absorption rate of the substance to be introduced into the skin.

There is thus a need for r f. skin treatment devices that are both non-invasive and provide effective and reproducible results for multiple treatment sites and multiple persons.

### SUMMARY OF THE INVENTION

An object of the invention is to provide a non-invasive treatment device using a radio-frequency electric current.

The object is achieved according to the invention by a non-invasive treatment device for heating an inner region of skin using an r.f. electric current, an outer surface region of the skin adjacent to the inner region being provided with a treatment substance selected to be transportable through the skin under influence of an electric field and further selected to increase the electrical transmission of the inner region, the device comprising:
a first treatment electrode configured and arranged to allow electric current to pass through an outer surface of the skin and through the inner region;
an r.f. generator configured and arranged such that, in use, an r.f. electric current generated by the r.f. generator is applied through the first treatment electrode to the outer surface of the skin and through the inner region;
a first field electrode configured and arranged to provide, in use, an electric field in the skin for transporting the treatment substance towards the inner region;
an electric field generator configured and arranged such that, in use, a DC electric field generated by the electric field generator is applied through the first field electrode to the treatment substance, whereby at least a portion of the treatment substance is transported from the outer surface region to the inner region; and
a control unit configured and arranged to control the r.f. generator and the electric field generator such that, in use, in a first treatment phase the control unit activates the electric field generator and maintains the r.f. generator in a non-active condition so that the DC electric field generated by the electric field generator transports the treatment substance from the outer surface region to the inner region during said first treatment phase, and in a second treatment phase, after the first treatment phase, the control unit activates the r.f. generator to apply the r.f. electric current through the first treatment electrode to the inner region during said second treatment phase.

The invention is based on the insight that the r.f. energy or current takes the path of least resistance through the tissue. Continuously rinsing the electrodes with saline solution, as proposed in the article by Abitabile, only affects the conductivity of the skin close to the electrodes. However, the path of the r.f. between the electrodes is determined by the impedance of the skin along the whole r.f. path. In skin rejuvenation and tightening applications, it is desirable to direct the treatment energy as precisely as possible to avoid damage to surrounding tissues. The treatment device according to the invention does this by providing, in a first treatment phase, a treatment substance to the inner region of the skin that increases the electrical transmission of the skin for the r.f. electrical current at the position in the skin where heating is to take place—i.e. the inner region. This first treatment phase is before a second treatment phase wherein subsequently the r.f. electric current is applied to the same inner region of the skin. For this purpose the treatment device according to the invention comprises a control unit configured and arranged to control the r.f. generator and the electric field generator such that the control unit activates the electric field generator is said first treatment phase, wherein the control unit maintains the r.f. generator in a non-active condition. In the second treatment phase, after the first treatment phase, the control unit activates the r.f. generator, so that the r.f. electric current is applied when the inner region has obtained an increased electrical conductivity for the r.f. electrical current.

The region to be treated may coincide with the inner region. Alternatively, it may be adjacent to the inner region—this may be advantageous when the region to be treated is a poor conductor of r.f., as the heat from the inner region will diffuse to the adjacent treatment region.

The treatment substance is selected to be transportable from an outer surface of the skin to the inner region under the influence of an electric field. The treatment device is configured and arranged to provide an electric field that transports, in use, the treatment substance to the inner region.

Typically, the field will be a DC-field or have a substantial DC-component. In this way, the device may direct the treatment substance to the path of the r.f. electric current through the first treatment electrode and through the inner region. The DC-field may also be pulsed to reduce undesirable irritation in the skin or even to prevent burns. When the r.f. electric current is applied in the second treatment phase, it flows through the path of increased transmission, obtained by reducing the electrical impedance of the desired path through the inner region in the first treatment phase.

The treatment device may be used as a monopolar device with a first external electrode, which typically is a mat placed under the person being treated, or a conductive pad or conductive plaster applied to the skin. It may also be a conducting probe inserted into a body cavity or penetrating into tissue. The function of the first external electrode may also be performed by a suitably grounded conductor, which, in combination with an appropriate configuration of the treatment device, will provide a flow of r.f. electric current from the first treatment electrode, through the skin, to ground.

Similarly, a second external electrode may be provided, which, in combination with an appropriate configuration of the treatment device, will provide a DC field which extends from the first field electrode, through the skin, to the second external electrode.

The first and second external electrodes may be electrically coupled so as to form a single combined external electrode. The treatment device may be further configured to provide a connection for either or both of the external electrodes, configured and arranged to connect the external electrode(s) to the r.f. generator and/or the field generator.

In a preferred embodiment of the treatment device according to the invention the control unit is configured and arranged such that, in use, the control unit maintains the electric field generator in a non-active condition during the second treatment phase. In this embodiment the electric field generator is active in the first treatment phase until, for example, an optimal electrical conductivity of the skin tissue for the r.f. electrical current is achieved. Subsequently, in the second treatment phase the electric field generator is not active during the application of the r.f. electric current, so that the optimal electrical conductivity is maintained during the application of the r.f. electric current.

It may be advantageous for the treatment device to further comprise a second treatment electrode connected to the r.f. generator, wherein the treatment device is further configured and arranged such that, in use, the r.f. electric current travelling from the first treatment electrode (50, 350) to the second treatment electrode is transmitted through the outer surface of the skin. A second treatment electrode comprised in the device (a bipolar device) provides a better defined r.f. electric current path through the skin, and simplifies the use of the device because no external electrode or ground plane is required for the r.f. current.

It may also be advantageous for the device to further comprise a second field electrode connected to the electric field generator, wherein the treatment device is further configured and arranged such that, in use, the DC electric field extends from the first field electrode through the inner region of the skin to the second field electrode. A second field electrode comprised in the device provides a better defined electric field through the skin, and simplifies the use of the device because no external electrode or ground is required for the DC field.

A further simplification of the construction of the treatment device is provided by electrically coupling the first treatment electrode and the first field electrode into a first combined electrode. This may provide a higher degree of coincidence between the path of the r.f. electric current and the path of the DC field. It may be provided in both monopolar and bipolar treatment devices.

The bipolar embodiment of the treatment device may be further simplified by electrically coupling the second treatment electrode and the second field electrode so as to form a second combined electrode.

It may be even more advantageous for the treatment device to further comprise a treatment substance supply configured and arranged to provide, in use, the treatment substance to the outer surface region of skin. This allows the treatment substance to be delivered in close proximity to the treatment device. The proximity is even more advantageous if the first field electrode or the first treatment electrode (or both) comprise an opening which is in communication with the treatment substance supply, the opening being arranged to supply, in use, the treatment substance to the outer surface region of skin.

For embodiments comprising a treatment substance supply, it may be advantageous if the device is further configured and arranged such that the first field electrode or the first treatment electrode comprises, in use, a volume of treatment substance between the treatment device and an outer layer of the skin. By using the electrical transmission properties of the treatment substance and retaining a volume of treatment substance between the device and the skin, a low impedance contact is made with an outer layer of the skin. This contact, being a transportable substance, may be fluidic enough to adapt its form to provide an optimum contact surface area, even for areas where the skin is rough. The volume of treatment substance may be retained by providing a surplus of treatment substance before activating the DC transportation field, or by supplying treatment substance at a higher flow rate than the rate of transport into the skin. Similarly, the volume of treatment substance may also be comprised in the first combined electrode, wherein the first field electrode and the first treatment electrode are electrically coupled.

It may be advantageous if the treatment device further comprises an impedance measurement circuit, connected to the first treatment electrode, and configured and arranged to measure, in use, the impedance of a path of the r.f. electric current through the skin. A lower impedance would allow an increase of the transmission of the r.f. electric current through the skin. As the treatment substance is transported through the skin, towards the inner region, the path of the r.f. electric current may change due to the changing transmission profile of the skin tissue through which it flows. The correspondence between the impedance measurement and the impedance of the path of the r.f. electric current through the inner region may be predicted using computer modeling. However, in many cases, it may be sufficiently accurate, if the impedance measurement is monitored, to determine whether a threshold has been passed and, if so, the r.f. electric current is provided by the treatment device.

So, by monitoring this impedance value, the progression of the treatment may also be monitored, or the presence of sufficient treatment substance at the inner region may be detected, indicating that treatment (or a further treatment step) may begin.

If the impedance measurement circuit is further connected to the control unit, the control unit may be configured and arranged such that, during the first treatment phase, the control unit controls at least one parameter of the DC electric field generated by the electric field generator in accordance with the impedance measured. Suitable parameters may be the time that the DC electric field is applied, the strength, the direction, and the polarity of the DC electric field. This may be used to control, inter alia, the speed of transport of the treatment substance through the skin, or to stop the DC electric field when the required portion of the treatment substance has reached the inner region, or to transport the treatment substance in the reverse direction towards the outer surface region.

If the treatment device comprises a treatment substance supply comprising a supply controller, the impedance measurement circuit may be connected to the supply controller and the supply controller may be configured and arranged to control at least one parameter of the treatment substance supply in accordance with the impedance measured. Suitable parameters may be the time that the substance is applied, the flow rate, the pressure, or the direction of the flow of the substance. Multiple substances may be used with different properties, so that a further parameter could be the type or concentration of the substance.

If the impedance measurement circuit is connected to the control unit, the control unit may be configured and arranged such that, during the second treatment phase, the control unit controls at least one parameter of the r.f. electrical treatment current in accordance with the impedance measured. Suitable parameters may be the time that the current is applied, the voltage, the frequency, the pulse duration, the duty cycle, and the strength of the electric current to be applied.

The treatment device may be advantageously used in the treatment of skin conditions, in particular wrinkles, acne, actinic keratosis, blemishes, scar tissue or discoloration.

### BRIEF DESCRIPTION OF THE DRAWINGS

FIG. 1 diagrammatically shows a first embodiment of the non-invasive treatment device in use when treating skin,

FIG. 2 shows a second embodiment of the non-invasive treatment device in use when treating skin,

FIG. 3 shows a third embodiment of the non-invasive treatment device in use when treating skin,

FIG. 4 shows a fourth embodiment of the non-invasive treatment device in use when treating skin,

FIG. 5A and FIG. 5B depict two examples of a field and/or treatment electrode comprising a volume of treatment substance, and

FIG. 6A and FIG. 6B depict examples of alternative electrode geometries.

It should be noted that items which have the same reference numbers in different Figures, have the same structural features and the same functions, or are the same signals. Where the function and/or structure of such an item has been explained, there is no necessity for repeated explanation thereof in the detailed description.

### DETAILED DESCRIPTION OF EXAMPLES

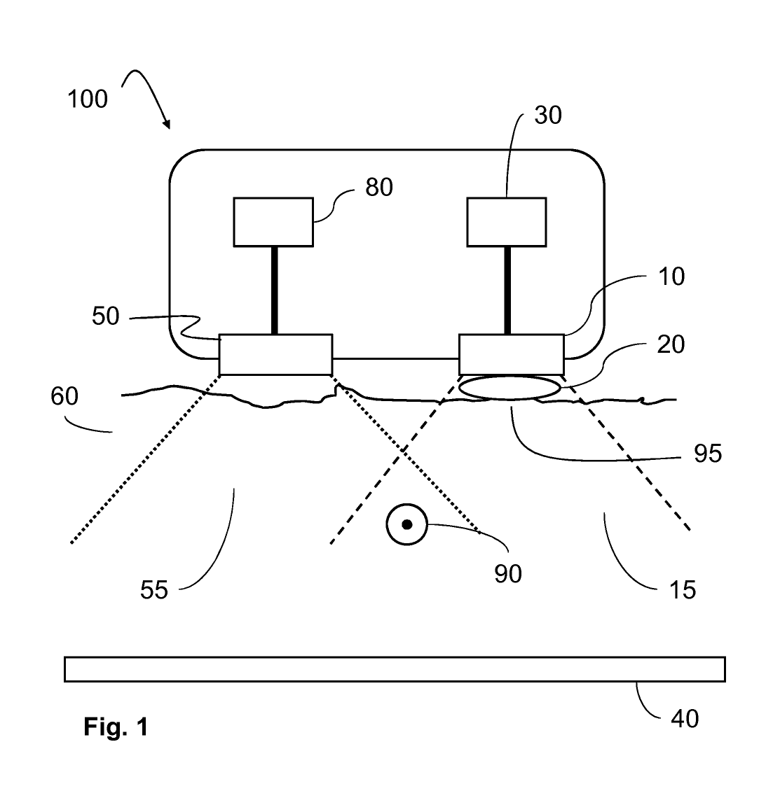

FIG. 1 schematically shows a first embodiment of a non-invasive skin treatment device 100 comprising a first treatment electrode 50 and an r.f. generator 80 connected to the first treatment electrode 50. The device 100 also comprises a first field electrode 10, connected to an electric field generator 30. The device 100 is configured and arranged to be brought into close proximity of human or animal skin 60, with the first treatment electrode 50 and the first field electrode 10 facing the outer layer of the skin. The first treatment electrode 50 is suitable for providing r.f. electric current in a first 55 region of the skin 60 and may make physical contact with the outer layer of the skin, or may be disposed at a small distance from the skin. The first field electrode 10 is suitable for providing an electric field in a second 15 region of the skin. An inner region 90 of the skin is the skin region that the user wishes to heat using r.f. and that is situated in a region of overlap between the first 55 and second 15 regions. The inner region 90 may be situated, inter alia, in the epidermis or dermis of the skin. The region which the user wishes to treat may lie on the r.f. current path through the skin, or immediately adjacent to that path. The heat generated in the skin by the r.f. current may diffuse to adjacent tissue regions. Treatment of multiple regions, both contiguous and non-contiguous, is also possible.

During use, the r.f. generator 80 generates an r.f. current, which may be used to heat the skin. The first treatment electrode 50 allows the r.f. current to pass through an outer surface of the skin and travel to the first region 55 of the skin.

The first treatment electrode 50 is usually suitable for making direct contact with the outer layer of the skin during use, whereby an electric current passes into the skin. Typically, a conductive substance, such as a gel, is used to reduce any contact impedance between the first treatment electrode 50 and the outer layer of the skin.

The r.f. signal for treatment may be an AC waveform, with a frequency in the range 0.5-50 MHz and a power in the range 1-400 W. A typical frequency used is 0.5-1 MHz, with a power of 25-100 W. The voltage and current to be used depend upon, inter alia, the treatment being performed and the depth of the inner region 90 below the outer layer of the skin. During use, the r.f. current is applied to the outer layer of the skin, and the current passes through skin tissue—the current heats the tissue directly along the r.f. current path including the inner region 90, and heats surrounding tissues indirectly by exposing them to fluids heated by the r.f. current. The electrical parameters may therefore be predetermined or controlled to provide the desired treatment current through the inner region 90, taking into account the expected range of skin impedances. The above-mentioned article by Faes discloses that the measured impedance of human skin can vary by an order of magnitude in the MHz range commonly used for r.f. The impedance is mainly comprised of resistance at lower frequencies, with a larger reactance contribution at higher frequencies. The impedance of the skin therefore depends not only on, inter alia, the type of skin tissue, the current path length, the position on the individuals body, and the individual, but also on the frequency of the r.f. current.

The main cause of inter-individual and intra-individual variation in skin impedance is believed to be due to variation in skin hydration and composition, particularly in the stratum corneum, and variations in skin thickness and structure. For example, the neck is generally more hydrated than the face, while within the face the chin has a higher hydration level. Natural hydration of the periorbital area significantly varies with age. This is further explained in the article “Biophysical parameters of the skin map of human face, regional and age-related differences” by S. Marrakchi and H. I. Maibach, Contact Dermatitis, 57 (2007) 28-34.

It is also known that the natural moisturizing factor of the skin decreases with photo-aging and intrinsic aging. As disclosed in the article “Comparative study of the hydration of the stratum corneum between four ethnic groups: influence of age” by S. Diridollou, J. de Rigal, B. Querleux, F. Leroy and V. H. Barbosa, International Journal of Dermatology, 46 (2007) 11-14; ethnicity and skin color may also play a role in skin hydration differences: sun-exposed skin of lighter pigmented skin types show more dehydration compared to non-exposed skin.

Another factor is the thickness and structure of the upper stratum corneum layer of the skin that highly varies among individuals and anatomical sites, and even within the same anatomical site. This is further explained in the article “Number of cell layers of the stratum corneum in normal skin—relationship to the anatomical location on the body, age, sex and physical parameters” by Z. Ya-Xian, T. Suetake, H. Tagami; Archives of Dermatological Research, 291 (1999) 555-559.

The path of the r.f. current through the skin is determined, inter alia, by the position, geometry and size of the first treatment electrode 50, and the point of contact of the first treatment electrode 50 on the outer layer of the skin. The actual path during treatment will be situated within the first region 55 of the skin where the r.f. treatment current may flow.

In the first embodiment of FIG. 1, the treatment device is monopolar—it only comprises a single r.f. electrode. An external r.f. electrode 40 may be convenient—typically this is a conductive pad applied to an outer surface of the skin, a conducting mat underneath the person being treated, or even an electrode which may be inserted into the skin at a convenient point as described in the Mulholland article mentioned above. The geometry, position and distance from the first treatment electrode may also influence the location of the path through which the r.f. current flows and the impedance of that path. The external r.f. electrode 40 may be electrically connected to the treatment device, or grounded, and in the treatment device an appropriate connection to ground is also made.

Smaller influences on the r.f. current path may be due to, inter alia, the area of contact between the first treatment electrode and an outer layer of the skin and the impedance of the different tissue types through which the r.f. current flows.

The dimensions of the first treatment electrode 50 may be in the range 0.1-5 cm. The dimensions may be varied depending on the type of treatment—typically electrodes of 0.1-1 mm are used for ablative or non-ablative skin rejuvenation; and 5 mm-10 mm for skin tightening. The first treatment electrode 50 may be circular or rectangular. It may also be an array of smaller electrodes which are typically used for a fractional treatment.

During use, the electric field generator 30 provides a DC electric field, or an electric field having a relatively high DC component, which is applied to the skin through the first field electrode 10. The first field electrode 10 does not need to make electrical or physical contact with the skin—it is configured to direct the electric field so as to enter the second region 15 of the skin and travel towards the inner region 90.

The direction and extent of the DC electric field through the skin is determined, inter alia, by the position, geometry and size of the first field electrode 10 and the electric potential between the first field electrode 10 and either an external electrode 40 or a ground plane. In the embodiment of FIG. 1, the external electrode for the r.f. treatment current and the external electrode for the field are electrically coupled so as to form a combined external electrode 40. As with the r.f. treatment current, the device 100 may also be used with a suitable ground plane and suitable ground connections in the device 100.

The device 100 is configured and arranged to be used with an appropriate treatment substance 20. In FIG. 1, the treatment substance 20 is depicted as disposed between the first field electrode 10 and an outer layer of skin 60. This treatment substance 20 may be comprised in, inter alia, a composition, a mixture, a solution, a suspension, a liquid, a serum, a cream or a gel. The treatment substance 20 may be provided before treatment using the treatment device 100, or during the treatment. The delivery of the treatment substance 20 may be performed using a separate device, or it may be comprised within the treatment device 100.

The treatment substance 20 is selected to be transportable through skin in the second region 15 under the influence of the DC electric field generated by the electric field generator 30 and may include inter alia, suitable molecules, ions, particles or a combination thereof. The treatment substance 20 is therefore selected to be susceptible to motion by a technique, such as inter alia, electrophoresis, electro-osmosis, iontopheresis or a combination thereof. The amount of DC current provided by the device through the first field electrode 10 depends upon the treatment substance 20 and the technique required to transport it.

Electrophoresis is a technique that involves the application of a DC electric field to a fluid comprising particles, whereby the particles move under the influence of the field. It may be used as a non-invasive transdermal technique to transport particles deeper into the skin.

Iontophoresis is the transport across the skin of ions or charged molecules, which may likewise also be used to transport ions deeper into the skin. Iontophoresis is generally applied to small molecules. Ionic solutions are preferred for iontophoresis, including NaCl, KCl, CaCl2, MgCl2. Other substances that increase the hydration level of the skin may also be suitable, such as water, humectants such as glycerin or urea, or emollients.

The DC current density should be in the range of 1-1000 micro-amps per square cm, preferably 50-200 micro-amps per square cm. The DC current should be applied through the first field electrode 10 for a period of time that is long enough to significantly lower the resistance of the skin, preferably at least 1 second, but up to 1 minute would also be acceptable for most users.

Electro-osmosis is the transport of an electrolyte through the narrow channels of an insulator. It may also be used for transporting ions deeper into the skin, as described in the article “The role of electro-osmotic flow in transdermal iontophoresis” by M J Pikal, Adv Drug Deliv Rev. 2001 Mar. 1;46(1-3):281-305.

The device 100 further comprises a control unit (not shown in the Figures) which is configured and arranged to control the r.f. generator 80 and the electric field generator 30. In particular, in use, in a first treatment phase the control unit activates the electric field generator 30 and maintains the r.f. generator 80 in a non-active condition, so that the DC electric field generated by the electric field generator 30 transports the treatment substance 20 from the outer surface region 95 to the inner region 90 during said first treatment phase. In a second treatment phase, after the first treatment phase, the control unit activates the r.f. generator 80 to apply the r.f. electric current through the first treatment electrode 50 to the inner region 90 during said second treatment phase. Thus, the device 100 applies the treatment substance 20 to an outer surface region 95 of the skin adjacent to the second region 15 before the application of the treatment r.f. electrical current. The control unit may be further configured and arranged such that, in use, the device 100 applies the treatment substance 20 to the second region 15 also during the application of the treatment r.f. electrical current in the second treatment phase. Alternatively, the control unit may be configured and arranged such that, in use, the control unit maintains the electric field generator 30 in a non-active condition during the second treatment phase. In this embodiment the electric field generator 30 is active in the first treatment phase until, for example, an optimal electrical conductivity of the second region 15 of the skin for the r.f. electrical current is achieved. Subsequently, in the second treatment phase the electric field generator 30 is not active during the application of the r.f. electric current, so that the optimal electrical conductivity is maintained during the application of the r.f. electric current. During use, the DC electric field generated by the electric field generator 30 is directed by the first field electrode 10 to extend towards the inner region 90 with an appropriate polarity and field strength. This will then transport at least a portion of the treatment substance 20 through the second region 15 to the inner region 90.

The first field electrode 10 may make electrical and/or physical contact with the treatment substance 20, but this is not necessary to direct the electric field to the treatment substance 20. Similarly, the first field electrode may make electrical and/or physical contact with an outer layer of the skin, but this is not necessary to direct the treatment substance 20 to the inner region 90.

In use, the treatment device is configured and arranged such that the first field electrode 10 faces the outer surface region 95 where the treatment substance 20 has been or will be supplied. The first region 15 where the electric field may be applied and the second region 55 where the r.f. current may flow do not need to coincide completely. The inner region 90, which the user wishes to heat, should be in a region of overlap between the first region 55 and the second region 15, and located such that the treatment substance 20 can penetrate the skin tissue sufficiently for the electrical transmission of the inner region 90 to be increased. The degree to which the first 55 and second 15 regions coincide is determined, inter alia, by the geometry and relative location of the field 10 and treatment 150 electrodes. The device 100 may also be configured to allow the user to modify such parameters to change the degree of coincidence. The device 100 may also be configured to allow the user to adapt the distance between the first treatment electrode and the first field electrode. Similarly, the device 100 may be provided with a skin position detector such that the treatment substance 20 is applied at a first skin position, the DC field is applied to this first skin position, and the r.f. current is applied to this skin position, wherein the user moves the device 100 relative to the skin.

The configuration of the DC field is determined to a significant degree by the treatment substance 20, and the required DC field to transport it through the skin. Any appropriate DC configuration may be used, such as current controlled, voltage controlled, pulsed current or voltage, positive polarity, or negative polarity. If the treatment device is to be operated with more than one treatment substances, the treatment device may advantageously comprise a current controller, a voltage controller, a pulse generator and/or a polarity switch.

The treatment substance 20 is further selected to increase the electrical transmission of the skin. Preferably, it is selected to increase the transmission of the inner region 90, so that after being transported by the electric field, the electrical transmission in the region of the skin to be heated is increased. This may be done by reducing the impedance of the tissue in that area or by increasing the conductance.

The process of iontophoresis, where a charged molecule is transported through the skin, is known from the article “Effects of iontophoresis on the electrical properties of human skin in vivo” by S. Y. Oh and R. H. Guy, International J of Pharmaceutics, 124 (1995) 137-142. A current density of 100 micro-Amps per square cm was used to deliver a 0.154M NaCl solution—the decrease in skin resistance was only measured to monitor the extent of iontophoretic delivery. The results showed that the skin resistance decreased by 90% within 10 seconds.

The invention is based on the insight that iontophoresis can rapidly lower the electrical resistance of the skin. This is particularly advantageous in combination with a treatment using r.f. current, because this will also quickly reduce the skin impedance for the r.f. current. The use of a treatment substance may allow deeper penetration of r.f. energy into the skin due to the decreased tissue impedance.

The conventional solution to the problem of variable skin conductance is to pre-treat the skin with a topical formula that passively diffuses through the skin. For example, the home-use TriPollar STOP device from Pollogen Ltd (www.stop-age.com) employs a hydrating gel containing glycerin with conducting salt. The disadvantages of a passive topical application step are that it can take hours for the formula to properly penetrate the skin and different skin types absorb the formula at different rates and efficiencies. For example, waiting for 2 hours after applying a skin hydration formula can achieve a decrease in skin resistance of only 20-40% as described in the article by Oh and Guy mentioned above.

The treatment device 100 rapidly increases skin conductance in and around the inner region 90 by applying a suitable electric field to the treatment substance 20. The electrical transmission of the skin is increased in the first treatment phase immediately prior to the second treatment phase during which the treatment r.f. electrical current is applied.

As the treatment substance 20 is transported through the skin, regions of skin tissue will change in impedance, which may influence and change the path of the r.f. current. At least a portion of the treatment substance 20 will be directed by the DC electric field towards the inner region 90. However, some of the treatment substance 20 may diffuse away from the inner region 90, and in practice some of the treatment substance may remain on the outer surface region 95.

The rate of transport through skin tissue may vary depending on the individual, the depth in the skin, the treatment substance used, the configuration of the treatment device and the area of the body to be treated. A very dry and dehydrated stratum corneum may be a significant cause of reduced transmission of r.f. into deeper skin tissue layers. To provide sufficient r.f. current to the inner region 90, the treatment device must be operated in a pre-determined or controlled way. In the simplest embodiments, the DC field may be activated with a certain strength and direction for a certain period of time. In more complicated embodiments, the parameters of the treatment device may be operated according to a predetermined or controlled algorithm—a predetermined algorithm may be determined by using appropriate computer modeling, whereas a controlled algorithm may be adapted during use by providing the treatment device with appropriate sensors. A high degree of control may be provided by including an appropriate microprocessor in the treatment device, configured and arranged to control the r.f. generation, the DC field generation and the treatment substance delivery.

Heating of the inner region 90 may be a combination of indirect and direct r.f. current flow through and/or adjacent to the inner region 90. Without wishing to be bound by theory, it is believed that the tissue surrounding the inner region 90, especially when located closer to the surface of the skin, would heat up more from r.f., because highly conductive tissue heats up but also directs r.f. towards less conductive tissue, which can heat up even more due to the high resistance. Computer modeling may be required to provide an accurate control algorithm for the treatment device. However, in many applications, simply turning on the DC field for a predetermined or controlled length of time may be sufficient to perform the treatment.

The treatment substance 20 may be comprised in a composition, a mixture, a solution, a suspension, a liquid, a serum, a cream or a gel that further comprises a further substance. This further substance may be, inter alia:
a conductive substance that substantially remains on the outer layer of the skin to reduce any contact impedance between the first treatment electrode 50 and the outer layer of the skin. In such a case, the further substance is selected to be less sensitive than the treatment substance 20 to be transported through the skin under the influence of an electric field.
a substance selected to provide a different effect at the inner region 90, such as denaturing or shrinking collagen, or promoting the healing of skin tissue following heat damage. The further substance is then also selected to be transportable through the skin under the influence of an electric field.
a substance selected to improve the transport and/or electrical transmission properties of the treatment substance 20.

It may also be advantageous to provide a plurality of treatment substances 20 these may be applied sequentially, or applied to separate areas on the outer layer of the skin in the regions where the electric field will influence the transport of these substances.

The same treatment substances 20 may also be applied sequentially in different concentrations. This provides a degree of control over the spatial profile of treatment substance 20 concentration within skin tissue—for example, using iontopheresis, a higher ionic-strength solution may be first transported at a higher current density, followed by transport of a lower ionic-strength solution.

During use, in the first treatment phase the electric field is applied with suitable parameters, such as strength, polarity and direction for an appropriate amount of time until a sufficient portion of the selected treatment substance 20 has been transported to the inner region 90. These parameters may be pre-determined by suitable experiments and/or computer modeling, and set by the user before use. Additionally or alternatively, the treatment device may be provided with a processor, programmed to execute an algorithm that controls these parameters during use.

The skilled person will also realize that the electric field does not need to be constant—for example, it may be advantageous to:
initially use a larger field strength to transport the treatment substance 20 through the outer layer of the skin, and then subsequently reduce the field strength for the rest of the transport;
initially direct the field approximately perpendicularly to the skin 60 surface, and then subsequently direct the field at a different angle for the rest of the transport;
initially use a first polarity to transport the substance 20 to the inner region 90, and then subsequently use the opposite polarity to transport the treatment substance 20 back towards the surface of the skin. This may be advantageous if the amount of treatment substance 20 at the inner region 90 is too large.

It may be advantageous for the treatment device to further comprise an impedance measurement circuit, connected to the first treatment electrode 50, and configured and arranged to measure, in use, the impedance of the r.f. current path through the skin. This impedance measurement may be used to control a parameter of the DC field and/or a parameter of the delivery of the treatment substance. For this purpose, the impedance measurement circuit may be connected to the control unit, and the control unit may be configured and arranged such that, during the first treatment phase, the control unit controls at least one parameter of the DC electric field generated by the electric field generator 30 in accordance with the measured impedance. A high impedance value may be used to provide an increased transport of treatment substance, and a low value may be used to reduce the transport of the treatment substance.

As this impedance will be dependent on the presence of the treatment substance 20 in the r.f. current path, the device may further comprise indicator lamps to display to the user whether the inner region 90 has a high enough electrical transmission for the treatment r.f. current to be applied. In practice, the correspondence between the impedance measured in the skin and the impedance of the r.f. current path may be determined experimentally, or by computer modeling. The impedance measurement will typically reflect the highest resistance in the tissue, usually in the most superficial layers. Similarly, during application of the r.f. current, the device may also indicate that the electrical transmission has become too low due to migration of the treatment substance away from the inner region 90.

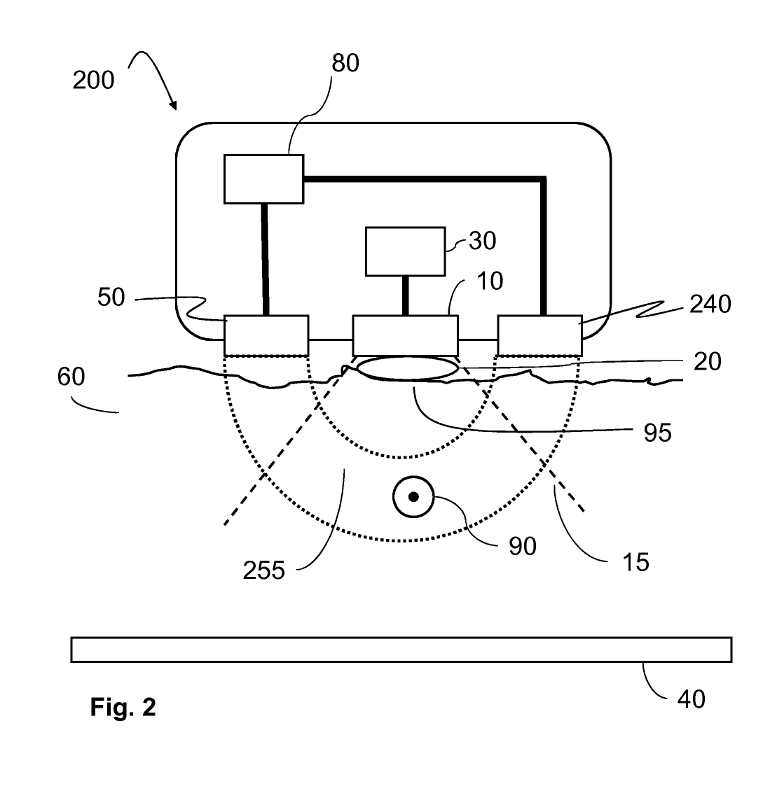

FIG. 2 depicts a second embodiment of the treatment device 200, having the following differences compared to FIG. 1:
the treatment device 200 further comprises a second treatment electrode 240, connected to the r.f. generator 80. This is often referred to as a bipolar device, as the device 200 comprises both the first treatment and the second treatment electrodes. In a monopolar device, by contrast, the function of the second treatment electrode is replaced by an external electrode or ground plane.
the treatment device 200 is further configured and arranged such that, in use, the radio-frequency electric current is transmitted through an outer surface of the skin to the second treatment electrode 240, and
the first field electrode 10 is disposed between the first treatment electrode 50 and the second treatment electrode 240.

Compared to the monopolar device of FIG. 1, the bipolar device of FIG. 2 may provide more control over the r.f. current path as the distance that the r.f. current flows may be shorter. The energy depth profile may depend on, inter alia, the distance between the first treatment electrode 50 and the second treatment electrode 240, and the size and geometry of the r.f. electrodes. The r.f. current will flow in a region 255 configured and arranged to comprise the inner region 90. It may be particularly advantageous for treatment locations close to the surface of the skin 60. For example, when treating skin tissue at superficial depths in combination with the transport of an ionic solution using iontopheresis, the ionic strength of the solution may be relatively high (above 0.1 M cations or anions, for example) and/or the solution may be delivered at a high current density (e.g. >50 microAmps per square cm) for a short delivery time (e.g. <10 s).

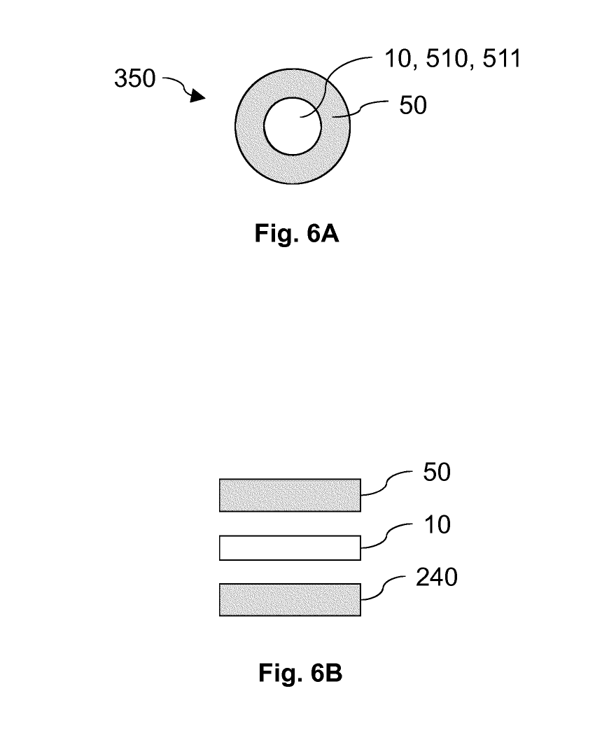

The first field electrode 10, treatment electrode 50 and/or second treatment electrode 240 may be circular or elliptical in cross-section. They may also be elongated in the cross-sectional plane substantially parallel to the surface of the skin being treated, as shown in FIG. 6B. An elongated field electrode 10 is disposed between an elongated treatment electrode 50 and an elongated second treatment electrode 240, the electrodes being elongated in parallel directions in the cross-sectional plane parallel to the skin during treatment. The use of elongated electrodes may allow a larger region 255 of skin to be treated without moving the treatment device.

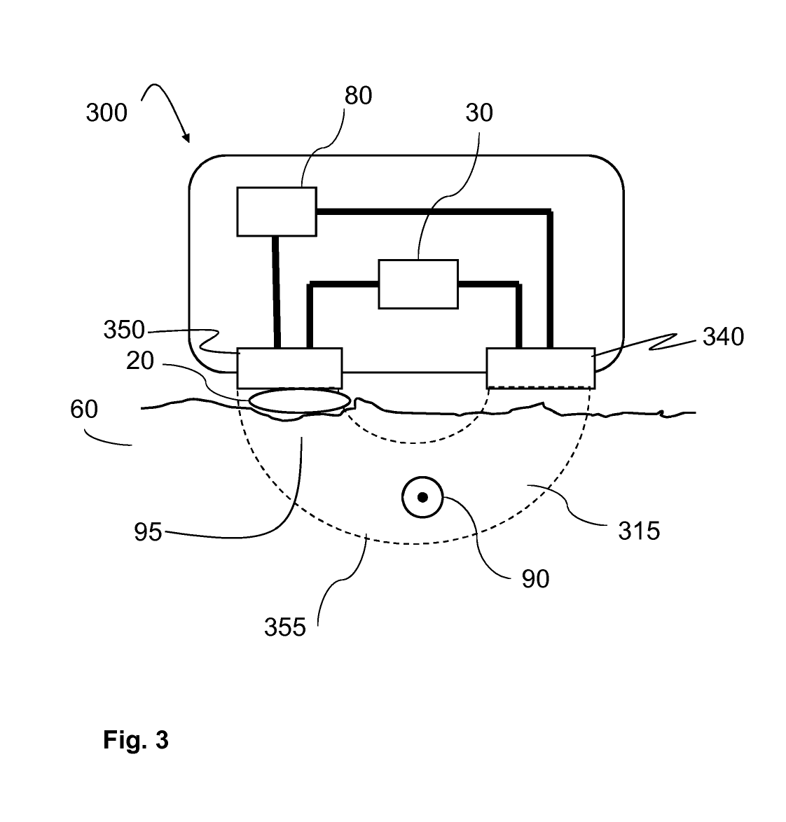

FIG. 3 depicts a third embodiment of the treatment device 300, having the following differences compared to FIG. 1:
no external electrode 40 or ground plane is required in use,
the first field electrode and the treatment electrode are electrically coupled so as to form a first combined electrode 350,
the treatment device 300 further comprises a second combined electrode 340, comprising a second field electrode electrically coupled to the second treatment electrode, the second combined electrode 340 being connected to the electric field generator 30, and
the treatment device 300 is further configured and arranged such that, in use, the DC electric field extends through the first combined electrode 350, through the skin, to the second combined electrode 340.

The first combined electrode 350 may functionally comprise treatment 50 and field 10 electrodes that are not distinct physically. In other words, the first combined electrode 350 is the same conducting body electrically connected to both the electric field generator 30 and the r.f. generator 80. Alternatively, the first combined electrode 350 may comprise treatment 50 and field 10 electrodes that are physically distinct, but electrically coupled. The first combined electrode 350 may also be physically distinct treatment 50 and field 10 electrodes that are electrically isolated, but attached to each other.

Compared to the bipolar device of FIG. 2, the bipolar device of FIG. 3 may provide a higher degree of coincidence between a region 355 where the r.f. treatment current may flow and a region 315 where the electric field may be provided. The area to be treated, in other words the inner region 90, may be greater in the embodiment of FIG. 3.

The first combined electrode 350 and second combined electrode 340 may be round, elliptical or elongated in cross-section in a plane substantially parallel to the skin surface when in use. The electrodes may comprise two distinct parts that have been electrically coupled, or constitute a single body having connections to the r.f. signal generator 80 and to the electric field generator 30.

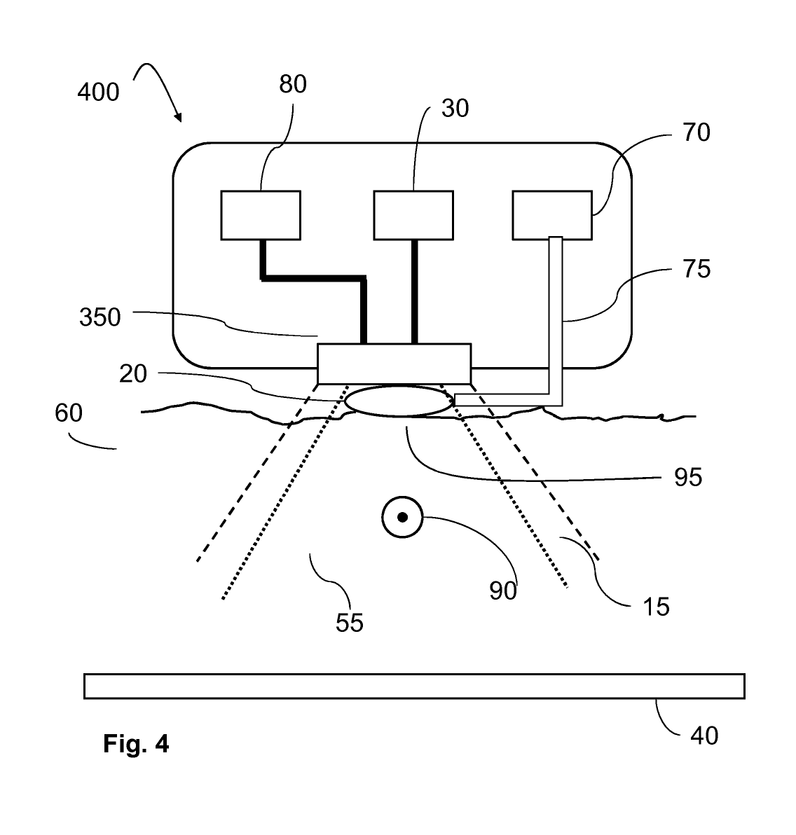

FIG. 4 depicts a fourth embodiment of the treatment device 400, having the following differences compared to FIG. 1:
the first field electrode and the treatment electrode are electrically coupled so as to form a first combined electrode 350,
the device 400 further comprises a treatment substance supply, comprising a supply controller 70 and a substance delivery part 75, configured and arranged to provide, in use, the treatment substance 20 to the outer surface region 95 of the skin. In other words, the treatment substance 20 is delivered between the first field electrode, which in this embodiment is comprised in the first combined electrode 350, and an outer layer of the skin.

During use, the treatment substance 20 is provided to the delivery part 75, which applies the treatment substance 20 in the required quantity to the outer surface region 95. By virtue of the delivery part 75 of the device 400, the treatment substance 20 may be provided proximate to the first field electrode 350. Control of the treatment substance is performed using the supply controller 70, which determines, inter alia, flow rate, pressure, dosage, and the moment of delivery.

The presence of treatment delivery part 75 in the device enables the treatment substance 20 to be applied at any point in time, in particular during the first treatment phase before the second treatment phase wherein the treatment r.f. electrical current is applied, or even during said second treatment phase. As the treatment device 400 is typically manually operated, this has the further advantage that the treatment substance 20 is always provided proximate to the first field electrode 450.

Optionally, the treatment substance supply may also comprise a tank or reservoir, connected to the delivery part 75, to provide sufficient treatment substance 20 for the treatment with r.f. current. It may also be advantageous to include a pump in the delivery part 75, but physical processes such as gravity and the capillary effect may also be employed to transport the treatment substance 20 through the delivery part 75.

If a plurality of treatment substances are to be provided, the treatment device 400 may also comprise further substance supplies. For example, different concentrations of treatment substance 20 may be provided by means of a further substance supply comprising a diluting medium, wherein the ratio of treatment substance 20 to diluting medium determines the concentration.

The treatment substance supply may comprise little more than the delivery part 75, with the supply controller 70 being provided in a separate device with a suitable connection between the treatment device 400 and this separate device. Although described only with respect to the fourth embodiment, the treatment supply 70, 75 may be provided in any of the other embodiments disclosed.

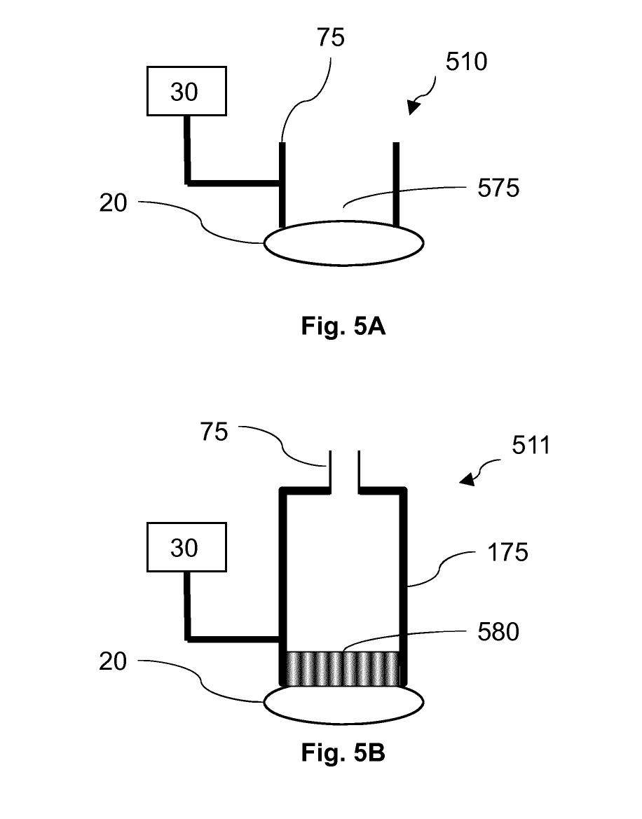

FIGS. 5A and 5B depict modified field electrodes 510, 511 for use in any of the embodiments comprising both a treatment substance delivery part 75 and a separate field electrode 10. In FIG. 5A, the first field electrode 510 comprises a section of the delivery part 75 having conductive walls connected to the electric field generator 30. The delivery part 75 opens into an opening 575, configured and arranged to provide, in use, treatment substance 20 to the outer surface region 95. The first field electrode 510 is further configured and arranged to electrically connect the treatment substance 20 via the conductive walls to the electric signal generator 30.

In use, any DC current that flows from the first field electrode 10, 510, 511 to the second field electrode or ground plane will pass through the treatment substance 20. DC current flows when the treatment substance 20 is selected to increase electrical transmission, for example when using an ionic solution, and transport into the skin is to be based on iontopherosis.

In other words, the first field electrode 10, 510, 511 comprises a volume of the treatment substance 20, which is fluidic, and which makes electrical contact with the skin. Such a fluidic electrode is advantageous because it may adapt its form depending on the profile of the outer surface of the skin. This optimizes the electrical contact cross-section, thus optimizing the contact resistance between the electrode and the skin, preventing undesired heating at the electrode contact. The volume of treatment substance may be retained in place by selecting the treatment substance 20 to have a pre-determined viscosity. It may also be advantageous to select the treatment substance 20 only for transport into the skin, and provide a mixture of the treatment substance 20 and a further substance via the treatment substance supply, the further substance being mainly selected for the purpose of being retained at the surface of the skin as part of the electrode, with a sufficiently high electrical transmission.

FIG. 5B depicts a further modified electrode 511, wherein the delivery part comprises a reservoir or tank 175 to retain a portion of the treatment substance 20. This reservoir 175 may be dimensioned such that it retains sufficient substance 20 to complete a treatment or it may have a buffer function to prevent interruption of the supply to the skin. The reservoir 175 has conductive walls connected to the electric field generator 30. The reservoir 175 opens into an opening comprising an absorbent material 580 such as a hydrogel matrix, swellable polymers, textile or fibrous material, configured and arranged to provide, in use, treatment substance 20 to the outer surface region 95. The absorbent material 580 provides a convenient delivery method when the modified field electrode 511 is brought into contact with the outer layer of skin.

Alternatively, instead of the conductive walls used, the delivery part 75 and/or reservoir 175 may comprise non-conducting walls and a connecting pin extending into the interior of the delivery part 75 or reservoir 175 such that the electric field generator 30 may be connected electrically to the treatment substance.

For embodiments with a separate treatment electrode 50, the modified field electrodes of FIG. 5A and 5B may be adapted for use as treatment electrodes as follows:
the connection to the electric field generator 30 is removed,
the modified treatment electrode is configured and arranged to electrically connect the treatment substance 20 to the r.f. generator 80, and
in use, the r.f. treatment current that flows from the first treatment electrode 50, 350 to the second treatment electrode or external electrode/ground plane will pass through the treatment substance 20.

As the treatment substance 20 is selected to increase electrical transmission, these modified treatment electrodes will provide improved electrical contact with the outer layer of the skin, thereby preventing charring. They also make it possible to first provide treatment substance 20 between the outer layer of the skin and the first treatment electrode 50, 350 before r.f. treatment current is applied.

As described above for the modified field electrode, a mixture may be provided comprising a further substance.

For embodiments where the first field electrode is comprised in a first combined electrode 350, the modified field electrodes of FIG. 5A and 5B may be adapted for use as combined treatment and field electrodes as follows:
the modified combined electrode is further configured and arranged to electrically connect the treatment substance 20 to both the r.f. generator 80 and the electric field generator 30,
in use, the r.f. treatment current that flows from the first treatment electrode 50, 350 to the second treatment electrode or ground plane will pass through the treatment substance 20, and
in use, any DC current that flows from the first field electrode 10, 510, 511 to the second field electrode or external electrode/ground plane will also pass through the treatment substance 20.

For embodiments where the first field electrode is comprised in a first combined electrode 350, a further alternative is depicted in FIG. 6A. FIG. 6A depicts a cross-section though a combined electrode 350 where the treatment electrode 50 and field electrode 10, 510, 511 are physically distinct. The cross-section is in a plane substantially parallel to the skin during use. The first treatment electrode 50 is an annulus, with the first field electrode 10, 510, 511 disposed at the centre of the annulus. The first treatment electrode 50 and first field electrode 10, 510, 511 are attached to each other—they may also be electrically coupled together, or electrically isolated from each other. In particular, the first field electrode may be one of the alternatives described in relation to FIGS. 5A and 5B.

During use, the treatment substance 20 is initially applied and transported into predetermined areas of the skin, using the DC electric field, during the first treatment phase. Subsequently, during the second treatment phase following the first treatment phase, the device treats the skin, using the r.f. electrical current. The user may perform a sliding motion of the device over a broad area of skin. By selecting appropriate settings of the r.f. generator 80, the device will only provide a treatment dose of sufficiently high r.f. current to those predetermined areas. As disclosed in the article by Oh and Guy mentioned above, the skin recovers its resistance, in as little as 10 min or up to 3 hr, after termination of iontophoresis. This means that the time frame to apply the r.f. treatment is similarly limited.

Although single electrodes are depicted and described, arrays of electrodes, either for the DC field or the r.f. treatment current or both, may also be used. For example, PCT application WO2008062365 describes an array of iontophoretic electrodes where geometry and coordination of electrode pairing influence the geometry and depth profile of the delivered substance in the skin.

The treatment devices according to the invention comprising arrays of electrodes may also be used to induce a fractional r.f. treatment by providing a plurality of field electrodes configured to be much smaller than the r.f. electrodes (for example, 200-700 microns), in a matrix configuration, where pairwise electrical synchronization may be used to avoid “crosstalk” during the application of the electric field. The r.f. current may then be applied, concentrating the r.f. energy delivery on the plurality of skin areas where the treatment substance was transported into the skin, resulting in fractional r.f. treatment.

It may be advantageous if the treatment device further comprises an impedance measurement circuit, connected to the first treatment electrode, and configured and arranged to measure, in use, the impedance of the radio-frequency current path through the inner region. A lower impedance would increase the electrical transmission of the radio-frequency current through the skin.

By measuring the impedance, the treatment device may determine the expected transmission of current through the skin. By monitoring this value, the progression of the treatment may be also be monitored or the presence of sufficient treatment substance at the inner region may be detected, indicating that treatment (or a further treatment step) may begin.

If the impedance measurement circuit is further connected to the control unit, the control unit may be configured and arranged such that, during the first treatment phase, the control unit controls or selects at least one parameter of the DC electric field in accordance with the impedance measured. Suitable parameters may be the time that the field is applied, the strength, the direction, and the polarity. This may be used to control, inter alia, the speed of transport of the treatment substance through the skin, or to stop the field when the required portion of the treatment substance has reached the inner region, or to transport the treatment substance in the reverse direction towards the outer surface region.

If the treatment device comprises a treatment substance supply comprising a supply controller, the impedance measurement circuit may be connected to the supply controller and the supply controller may be configured and arranged to control or select at least one parameter of the treatment substance supply in accordance with the impedance measured. Suitable parameters may be the time that the substance is applied, the flow rate, the pressure, or the direction. Multiple substances may be used with different properties, so a further parameter could be the type of substance.

If the impedance measurement circuit is connected to the control unit, the control unit may be configured and arranged such that, during the second treatment phase, the control unit controls or selects at least one parameter of the r.f. treatment current in accordance with the impedance measured. Suitable parameters may be the time that the current is applied, the voltage, the frequency, pulse duration and duty cycle, and the maximum current to be applied.

It may also be advantageous to measure impedance more directly by applying an AC current and measuring the phase shift and voltage drop between the first field electrode and the second field electrode or ground plane. More than one type of impedance measurement may be combined in the treatment device.

So, in summary, the invention provides a non-invasive r.f. current treatment device to be used with an agent or treatment substance 20, such as an ionic salt solution. The substance is transported in a first treatment phase using a DC electric field generated by the device, such as in iontophoresis, to move the treatment substance 20 into the skin so that the skin's electrical transmission for r.f. electrical current is increased. The path of any r.f. current is influenced by the distribution of the treatment substance 20 in the skin. Applying r.f. electrical current during a second treatment phase, after the first treatment phase, causes heating in skin tissues coinciding with the path of the r.f. electrical current, allowing skin tissue to be treated that is either coincident with the r.f. electrical current path, or immediately adjacent thereto. The region of skin treated with r.f. electrical current may be the upper tissue layers. This renders superfluous the conventional hydration step necessary for r.f. skin preparation, which is poorly controllable and highly variable depending on the anatomic site and the individual being treated. The lowered skin impedance enables well-defined r.f. delivery to skin tissues, and a high degree of control over the r.f. energy delivery depth profile. The invention also makes it possible to treat deeper layers, using the r.f. current, than traditional techniques due to the high penetration of the treatment substance 20 caused by application of the electric field.

It should be noted that the above-mentioned embodiments illustrate rather than limit the invention, and that those skilled in the art will be able to design many alternative embodiments.

In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer.

The word “module” should not be interpreted to mean that the functionality and hardware are distinguishable in the device. It is used to indicate a functionality that the device comprises, and in practice different “modules” may use some or all of the same hardware and optical components.

In the device claim enumerating several means, several of these means may be embodied by one and the same item of hardware. The mere fact that certain measures are recited in mutually different dependent claims does not indicate that a combination of these measures cannot be used to advantage.

### OVERVIEW OF REFERENCE NUMBERS

10 first field electrode (dc electric field)
15 Second region of skin where electric field may be disposed—^{st} and 2^{nd} embodiments
20 Treatment substance
30 Electric field generator
40 Return electrode for both field and r.f.
50 First treatment electrode (r.f. current)
55 First region of skin where r.f. treatment current may flow—1^{st} embodiment
60 Skin
70 Treatment substance supply controller (part of supply)
75 Treatment substance delivery part (part of supply)
80 r.f. generator
90 Inner region (of skin)—point to be treated
95 Outer surface region—point where treatment solutions are applied (on outside)
100 Treatment device—1^{st} embodiment—monopolar for treatment and field
200 Treatment device—2^{nd} embodiment—monopolar for field, bipolar for treatment
240 Second treatment (r.f.) electrode—comprised in bipolar device
255 First region of skin where r.f. treatment current may flow—2^{nd} embodiment
300 Treatment device—3^{rd} embodiment—bipolar for treatment and field, shared electrode
310 Field electrode comprising volume of treatment substance
315 Second region of skin where electric field may be disposed—3^{rd} embodiment
340 Second combined electrode—treatment and field
350 First combined electrode—treatment and field—bipolar
355 First region of skin where r.f. treatment current may flow—3^{rd} embodiment
400 Treatment device—4^{th} embodiment—monopolar, combined electrode
415 Second region of skin where electric field may be disposed—4^{th} embodiment
510 Modified separate first field electrode
511 Modified separate first field electrode with reservoir and absorbent material
550 Modified first combined electrode—field and treatment
575 Opening in electrode for substance delivery to skin
580 Absorbent material used in substance delivery
610 First field electrode—strip
615 Second field electrode—strip
640 Second treatment electrode—strip
650 First treatment electrode—strip
740 Second treatment electrode—multiple electrodes
750 First treatment electrode—multiple electrodes
840 Second treatment return electrode—rectangular
850 First treatment electrode—rectangular

1. A non-invasive treatment device for heating an inner region of skin using an r.f. electric current, an outer surface region of the skin adjacent to the inner region being provided with a treatment substance selected to be transportable through the skin under influence of an electric field and further selected to increase the electrical transmission of the inner region, the device comprising:
a first treatment electrode configured and arranged to allow electric current to pass through an outer surface of the skin and through the inner region;
an r.f. generator configured and arranged such that, in use, an r.f. electric current generated by the r.f. generator is applied through the first treatment electrode to the outer surface of the skin and through the inner region;
a first field electrode configured and arranged to provide, in use, an electric field in skin for transporting the treatment substance towards the inner region; and
an electric field generator configured and arranged such that, in use, a DC electric field generated by the electric field generator is applied through the first field electrode to the treatment substance, whereby at least a portion of the treatment substance is transported from the outer surface region to the inner region;
wherein the non-invasive treatment device further comprises a control unit configured and arranged to control the r.f. generator and the electric field generator such that, in use, in a first treatment phase the control unit activates the electric field generator and
maintains the r.f. generator in a non-active condition so that the DC electric field generated by the electric field generator transports the treatment substance from the outer surface region to the inner region during said first treatment phase, and in a second treatment phase, after the first treatment phase, the control unit activates the r.f. generator to apply the r.f. electric current through the first treatment electrode to the inner region during said second treatment phase.

2. The treatment device according to claim 1, wherein the control unit is configured and arranged such that, in use, the control unit maintains the electric field generator in a non-active condition during the second treatment phase.

3. The treatment device according to claim 1, wherein the treatment device further comprises a second treatment electrode connected to the r.f. generator, wherein the treatment device is further configured and arranged such that, in use, the r.f. electric current is transmitted to the outer surface of the skin from the first treatment electrode to the second treatment electrode.

4. The treatment device according to claim 1, wherein the treatment device further comprises a second field electrode, wherein the treatment device is further configured and arranged such that, in use, the DC electric field extends from the first field electrode through the inner region of the skin to the second field electrode.

5. The treatment device according to claim 1, wherein the first treatment electrode and the first field electrode are electrically coupled so as to form a first combined electrode.

6. The treatment device according to claim 3, wherein the treatment device further comprises a second field electrode, wherein the treatment device is further configured and arranged such that, in use, the DC electric field extends from the first field electrode through the inner region of the skin to the second field electrode, and wherein the second treatment electrode and the second field electrode are electrically coupled so as to form a second combined electrode.

7. The treatment device according to claim 1, wherein the device further comprises:
a treatment substance supply configured and arranged to provide, in use, the treatment substance to the outer surface region of the skin.

8. The treatment device according to claim 7, wherein the first field electrode or the first treatment electrode comprises an opening in communication with the treatment substance supply, the opening being arranged to supply, in use, the treatment substance to the outer surface region of the skin.

9. The treatment device according to claim 7, wherein the device is further configured and arranged such that the first field electrode or the first treatment electrode comprises, in use, a volume of treatment substance between the treatment device and an outer layer of the skin.

10. The treatment device according to claim 7, wherein the device is further configured and arranged such that the first field electrode and the first treatment electrode are electrically coupled so as to form a first combined electrode, the first combined electrode comprising, in use, a volume of treatment substance between the treatment device and an outer layer of the skin.

11. The treatment device according to claim 1, wherein the treatment device further comprises an impedance measurement circuit connected to the first treatment electrode, and configured and arranged to measure, in use, the impedance of a path of the r.f. electric current through the skin.

12. The treatment device according to claim 11, wherein the impedance measurement circuit is further connected to the control unit, and wherein the control unit is configured and arranged such that, during the first treatment phase, the control unit controls at least one parameter of the DC electric field generated by the electric field generator in accordance with the impedance measured.

13. The treatment device according to claim 11, wherein the device further comprises a treatment substance supply configured and arranged to provide, in use, the treatment substance to the outer surface region of the skin, the treatment substance supply comprising a supply controller, wherein the impedance measurement circuit is connected to the supply controller and wherein the supply controller is configured and arranged to control at least one parameter of the treatment substance supply in accordance with the impedance measured.
